(19) United States
(12) Patent Application Publication
(10) Pub. No.: US 2014/0128913 A1
(43) Pub. Date: May 8, 2014

(54) SURGICAL SYSTEM INCLUDING AN ANCHOR INSERTABLE INTO A CANNULA OF AN INTRODUCER

(71) Applicant: COLOPLAST A/S, HUMLEBAEK (DK)

(72) Inventors: Sarah J. Deitch, Minneapolis, MN (US); Michael M. Witzmann, Shoreview, MN (US)

(73) Assignee: COLOPLAST A/S, HUMLEBAEK (DK)

(21) Appl. No.: 14/050,352

(22) Filed: Oct. 10, 2013

Related U.S. Application Data
(60) Provisional application No. 61/721,563, filed on Nov. 2, 2012.
(60) Provisional application No. 61/884,142, filed on Sep. 30, 2013.

(51) Int. Cl. A61B 17/04

(52) U.S. Cl. CPC A61B 17/0401
USPC 606/232

(57) ABSTRACT

A surgical system includes an anchor and an introducer provided to deliver the anchor into tissue. The anchor includes a body having a pointed leading end that is configured to pierce the tissue, a spine projecting radially away from the body and having a width and a height configured to allow the spine to engage with the tissue, an eyelet attached to a trailing end of the body, and a length of suture attached to the eyelet. The introducer includes a cannula with a pointed distal end, an opening formed in the cannula that is sized to receive the body of the anchor, and a slot formed in a wall of the cannula at a distal end portion of the cannula and sized to receive the width of the spine.

BACKGROUND

Intracorporeal suturing of tissue during surgery presents challenges to the surgeon in that the surgeon is called upon to manipulate suturing instruments within the confines of a relatively small incision formed in the patient's body. In some cases, the surgeon is unable to see the suture site. In such a case, the surgeon will digitally palpate with a finger to locate a landmark within the intracorporeal site, and then deliver the suture near at or near the landmark. Tying of the suture inside the patient at the intracorporeal site can be challenging since the surgeon is unable to see the site.

Improved suturing instruments and improved methods of delivering sutures would be welcomed by the surgical staff.

### SUMMARY

One aspect provides a surgical system including an anchor and an introducer provided to deliver the anchor into tissue. The anchor includes a body having a pointed leading end that is configured to pierce the tissue, a spine projecting radially away from the body and having a width and a height configured to allow the spine to engage with the tissue, an eyelet attached to a trailing end of the body, and a length of suture attached to the eyelet. The introducer includes a cannula with a pointed distal end, an opening formed in the cannula that is sized to receive the body of the anchor, and a slot formed in a wall of the cannula at a distal end portion of the cannula and sized to receive the width of the spine. When readied for use, the anchor is secured in the introducer with the body of the anchor inserted into the opening of the cannula with the pointed leading end of the body located proximal of the pointed distal end of the cannula. The spine of the anchor is inserted into the slot of the cannula such that the anchor does not rotate relative to the cannula.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a perspective view of one embodiment of a surgical system including an anchor that is insertable into a cannula of an introducer.

FIG. 2A is a perspective view of the anchor illustrated in FIG. 1.

FIG. 2B is an end view of the anchor.

FIG. 2C is a perspective view of one embodiment of an anchor for the surgical system illustrated in FIG. 1.

FIG. 3 is a perspective view of the anchor outside of the cannula illustrated in FIG. 1.

FIG. 4 is a perspective view of the anchor inserted into a lumen of the cannula illustrated in FIG. 1.

FIG. 5 is a schematic view of one embodiment of the surgical system provided to anchor a support material to tissue of the human body, with the support material having an arm inserted through each of two obturator foramen of the pelvis.

FIG. 6 is a schematic view of one embodiment of the surgical system employed to anchor a support material to the tissue of the human body showing a pre-pubic portion being attached to the periosteum of the pubic bone.

FIG. 7 is a schematic view of the anchor illustrated in FIG. 1 secured to tissue with a stopper coupled with a suture and located between the anchor and a slip knot.

FIG. 8 is a schematic view of two anchors as illustrated in FIG. 1 secured to tissue and coupled with a suture.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

The features of the various exemplary embodiments described in this application are suitable and intended to be combined with each other, unless specifically noted otherwise.

Anterior means “forward” or “front,” and posterior means “rearward” or “back.” Relative to surfaces of an organ in the human body, an anterior surface of an instrument inserted into the organ will be oriented forward toward the belly and a posterior surface will be oriented rearward toward the spine.

End means an end most location and end portion means that segment adjacent to and near the end of an object. For example, two opposing ends of an object are each equidistant from a mid-point of the object and between the mid-point and each end of the object is an end portion of the object.

Embodiments provide a surgical system including an introducer that is configured to deliver an anchor to an intracorporeal tissue site. The introducer includes a cannula that allows placement of an anchor at a landmark in tissue deep within an incision site, which may be out of the field of vision of the surgeon. The anchor is configured to be secured within the cannula so that it does not rotate or fall out of the cannula during insertion into the tissue. A length of suture is provided that is attached to the anchor, where the suture may be tied or otherwise terminated to itself outside of the incision site and then subsequently directed to the intracorporeal landmark.

Some incontinence treatment devices have several arms, including some form of arms that traverse the obturator foramen (called transobturator arms) and other arms that are implanted anterior to the pubic bone (called pre-pubic arms). A first set of tools is used to place the transobturator arms and a second, different set of tools is used to place the pre-pubic arms. The pre-pubic arms are tunneled anterior to the pelvis and exit the skin of the abdomen.

In contrast, embodiments of the system described in this specification provide a support with two transobturator arms and a system to attach a portion of the support directly and efficiently to the periosteum tissue. The system obviates the use of additional pre-pubic arms and additional tools that tunnel the pre-pubic arms under the skin. The system is easier to implant compared to a four arm or six arm support, and reduces the amount of time that the patient is in the operating room.

One approach to treating urinary incontinence places a support inferior to the urethra and directs arms upward from the support alongside the bladder along a U-shaped pathway. A significant advance over the U-shaped pathway was provided by Dr Emmanuel Delorme as described in his U.S. Pat. No. 6,638,211 and included placing arms of a support through the obturator foramen along a V-shaped pathway. This application provides another advance in supporting the pelvic anatomy by recognizing that support material can be robustly attached to the periosteum tissue through the use of an anchoring system. The anchoring system allows the surgeon to place the support inside of the patient and directly fixate the support to periosteum tissue that is present over the exterior of the bones. This approach does away with needles and other tools that tunnel the arms of a support through tissue. The anchoring system described in this application is compatible with a true single (only one) incision formed in the patient.

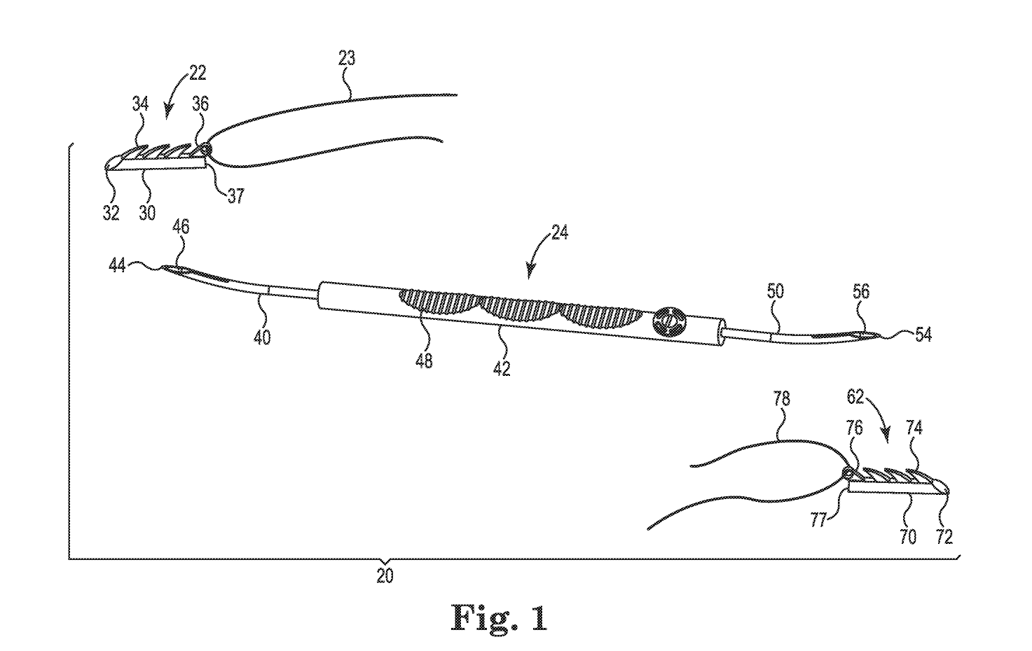

FIG. 1 is a perspective view of one embodiment of a surgical system 20. The surgical system 20 (system 20) includes an anchor 22 attached to a length of suture 23 and an introducer 24 adapted to deliver the anchor 22 to an intracorporeal landmark. The anchor 22 is sized to be inserted into the introducer 24, and the introducer 24 is sized to be inserted through a single incision to push or direct the anchor 22 into tissue. The suture 23 trails behind the anchor 22 and is available for subsequent ligation of the tissue, or for subsequent attachment of a support to the tissue.

The anchor 22 includes a body 30 having a pointed leading end 32 that is configured to pierce tissue, a spine 34 projecting radially away from the body 30 and configured to engage with or anchor to tissue, and an eyelet 36 attached to a trailing end 37 of the body 30. The length of suture 23 is inserted through the eyelet 36.

The introducer 24 includes a cannula 40 extending from a handle 42. The cannula 40 has a pointed distal end 44 and an opening 46 formed in the cannula 40. The opening 46 or lumen 46 is sized to receive the body 30 of the anchor 22. The handle 42 includes a gripping surface 48 formed on at least one side of the handle 42. It is acceptable to provide the handle 42 with several gripping surfaces or with no gripping surfaces. During a suturing procedure, the anchor 22 is loaded into the opening 46 of the cannula 40 and the surgeon grips the handle 42 and directs the pointed distal end 44 of the cannula to a targeted tissue landmark. Force delivered to the handle 42 in a distal direction will drive the pointed distal end 44 of the cannula 40 into the tissue, such that a subsequent withdrawal of the introducer 24 in a proximal direction will allow the introducer 24 to exit the tissue. The spine 34 (and in some cases the eyelet 36) engages with the tissue, thus leaving the anchor 22 engaged with and deposited in the tissue after the cannula 40 is withdrawn.

In one embodiment, the introducer 24 includes a pair of cannulas, including a second cannula 50 having a pointed distal end 54 and an opening 56 formed in the cannula 50. The second cannula 50 is provided to receive a second, separate anchor. With this in mind, a second anchor 62 is provided having a body 70 having a pointed leading end 72, a spine 74 projecting radially from the body 70, an eyelet 76 attached to a trailing end 77 of the body 70, and a second length of suture 78 attached to the eyelet 76. In this embodiment, the introducer 24 is operable to deliver the first anchor 22 out of the first cannula 40 and to subsequently deliver the second anchor 62 out of the second cannula 50. The gripping surface 48 is configured to allow the translation or rotation of the instrument to selectively move each of the cannulas 40, 50 to a forward facing proximal position.

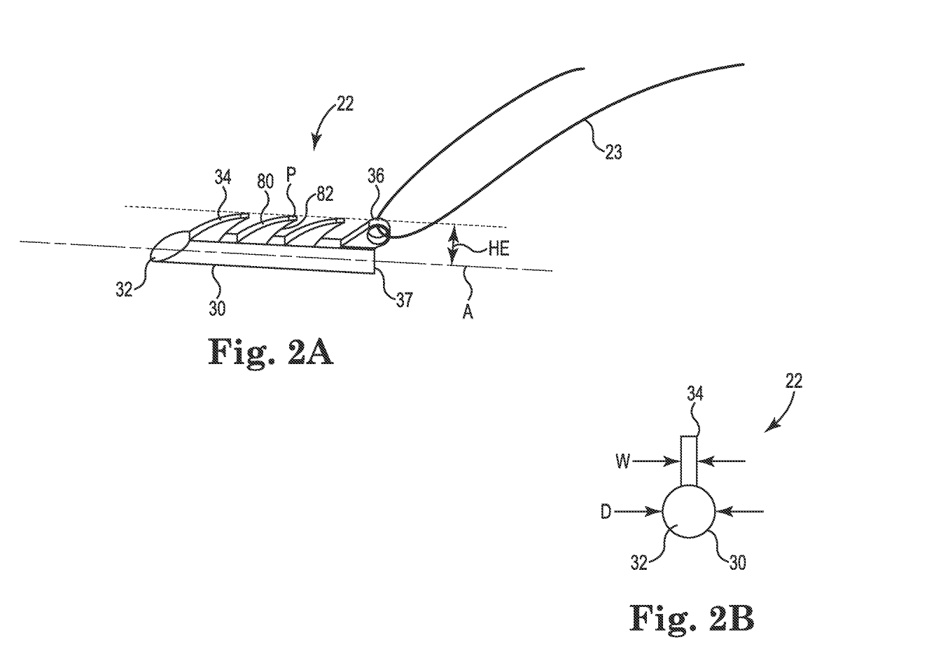

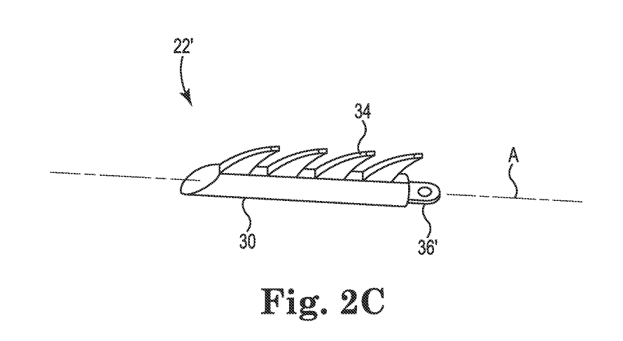

FIG. 2A is a perspective view of the anchor 22 and FIG. 2B is an end view of the anchor 22. The anchor 22 includes multiple spines 34 extending from the body 30. In one embodiment, the spines 34 project radially away from a center longitudinal axis A of the body 30, with each spine 34 shaped as a shark fin having a curved leading edge 80 that meets with a curved trailing edge 82 at a point P. The curved leading edge 80 is oriented to diverge away from the pointed leading end 32 of the body 30 to allow the anchor 22 to glide into tissue and prevent the anchor from pulling out of the tissue. Although three spines 34 and one eyelet 36 are illustrated, the anchor 22 is also suitably provided with a single spine 34 and one eyelet 36. The anchor 22 is also suitably provided with more than three spines 34.

The eyelet 36 projects radially away from the center longitudinal axis A of the body 30 and as such is also configured to engage with tissue. For example, the eyelet 36 is provided with a height HE that is substantially equal to the height of the spines 34 (the distance that the point P is away from the center axis A). The eyelet has a width substantially equal to the width W of the spine 34.

The body 30 of the anchor 22 is substantially circular in lateral cross-section (FIG. 2B). The anchor 22 is configured to slide in an entry direction through the tissue, and is shaped to prevent withdrawal of the anchor 22 in the direction that is opposite of the entry direction. The curved leading edge 80 of the shark fin shape of the spines 34 facilitate the easy sliding of the anchor 22 through the tissue in the entry direction, and the curved trailing edge 82 of the spines 34 configure the anchor to resist being pulled out of the tissue in the direction that is opposite of the entry direction. In one embodiment, the body 30 of the anchor 22 has a diameter D, and the spine 34 has a width W that is less than about 25% of the diameter D (FIG. 2B).

FIG. 2C is a perspective view of one embodiment of an anchor 22′ provided with an eyelet 36′that is disposed on the center longitudinal axis A of the body 30. The spines 34 of the anchor 22′ are provided to engage with tissue, and the eyelet 36′ is streamlined to follow the body 30 into the tissue channel that is formed when the anchor 22′ is driven into the tissue by the introducer 24 (FIG. 1).

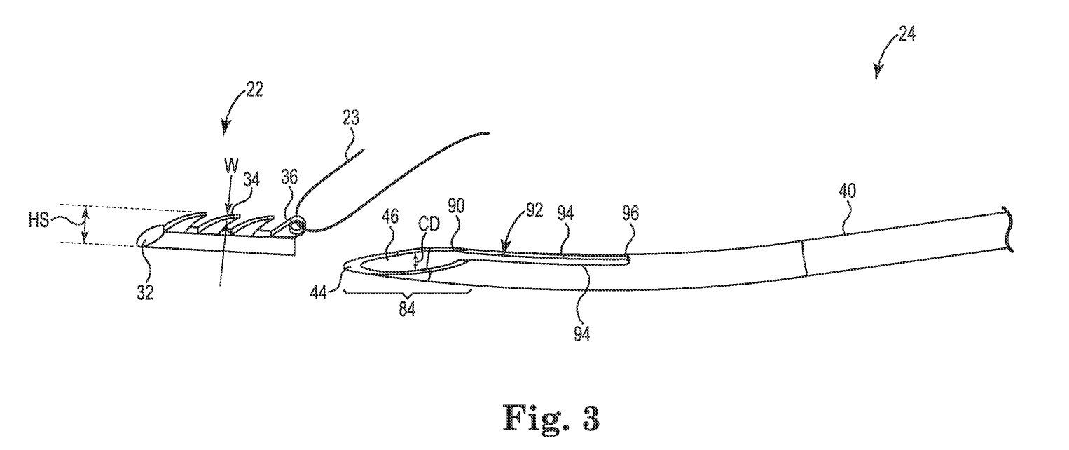

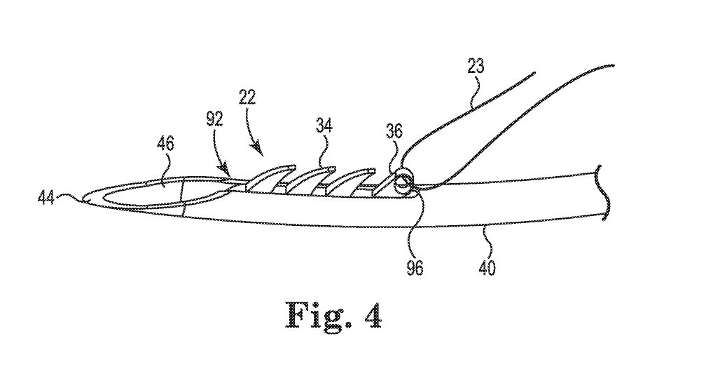

FIG. 3 is a perspective view of the anchor 22 positioned for insertion into the cannula 40 of the introducer 24 and FIG. 4 is a perspective view of the anchor 22 inserted into the cannula 40. The body 30 of the anchor 22 is sized to slide into the opening 46 (also called a lumen 46) of the cannula 40 with the spine 34 projecting out of the cannula 40. With reference to FIG. 3, the inside diameter of the lumen 46 of the cannula 40 provides a cannula diameter CD, and the spine 34 has a height HS that is greater than the cannula diameter CD. The height HS the spine 34 is at least 5% greater than the cannula diameter CD. For example, the height HS of the spine 34 is in the range of 5-100% greater than the cannula diameter CD.

It is acceptable for the height HE (FIG. 2A) of the eyelet 36 to be equal to the height HS of the spine 34. It is also acceptable for the height HE (FIG. 2A) of the eyelet 36 to be different from and not equal to the height HS of the spine 34.

The cannula 40 includes a tapered distal end portion 84 that tapers to the pointed distal end 44, where the tapered distal end portion 84 provides the cannula 40 with a needle-like point adapted for insertion through tissue. In some applications, the pointed distal end 44 of the cannula 40 is sharp and needle-like and is so configured to enter the periosteum tissue covering a boney surface and glide under the periosteum tissue and over the bone. In this manner, the cannula is configured to deliver the anchor 22 between the periosteum tissue and the bone.

The cannula 40 has a wall 90 that forms or defines the lumen 46 and a slot 92 formed through the wall 90. The slot 92 is proximal of the tapered distal end portion 84 and extends through the wall 90 to communicate with the lumen 46. The slot 92 includes a pair of opposed longitudinal side edges 94 that extend from a proximal lateral edge 96 in a distal direction to the distal end portion 84. The width of the slot between the longitudinal side edges 94 is sized to receive the width W of the spines 34. The cannula diameter CD is sized to receive the diameter D (FIG. 2B) of the body 30 of the anchor 22.

With reference to FIG. 4, when the anchor 22 is loaded into the cannula 40, the pointed leading end 32 of the body 30 is located proximal of the pointed distal end 44 of the cannula 40, and the spines 34 and the eyelet 36 extend outside of the cannula 40 and are positioned to engage with tissue during implantation of the anchor 22. The proximal lateral edge 96 of the slot 92 is positioned to push against the eyelet 36 and drive the anchor 22 into the tissue. The opposed longitudinal side edges 94 of the slot 92 provide a stanchion that restrains the spines 34 and prevents the anchor 22 from rotating relative to the cannula 40. The spines 34 and the eyelet 36 slide in a longitudinal direction relative to the slot 92 to allow the cannula 40 to be removed from the tissue while leaving the anchor 22 implanted.

Suitable materials for fabricating the anchor 22 include plastics, or metal, or sintered material. One suitable material for fabricating the anchor 22 is polypropylene. Another suitable material for fabricating the anchor 22 is a bioabsorbable polymer that configures the anchor 22 to be absorbed into the body over a period of several weeks.

Suitable materials for fabricating the length of suture 23 include bio-inert components that do not bioabsorb, or bioabsorbable components that are configured to be absorbed or resorbed by the body. One suitable material for fabricating the length of suture 23 is polypropylene. Other suitable materials for fabricating the length of suture 23 include dissolvable sutures available from Ethicon™, a J&J Company located in Somerville, N.J., and include Monocryl™ (polyglycaprone 25) sutures, coated Vicryl™ (polyglactin 910) sutures, Ethicon P1us™ Sutures, or polydioxanone sutures as examples.

Suitable materials for fabricating the cannula 40 and include plastics or metal. One suitable material for fabricating the cannula 40 is stainless steel. Other suitable materials are acceptable.

With reference to FIG. 1, the anchor 22 is useful for fixating a support material within a patient's body. The introducer 24 is sized to place the anchors 22 through a single incision and into the periosteum tissue that covers the pubic bone, examples of which are described below.

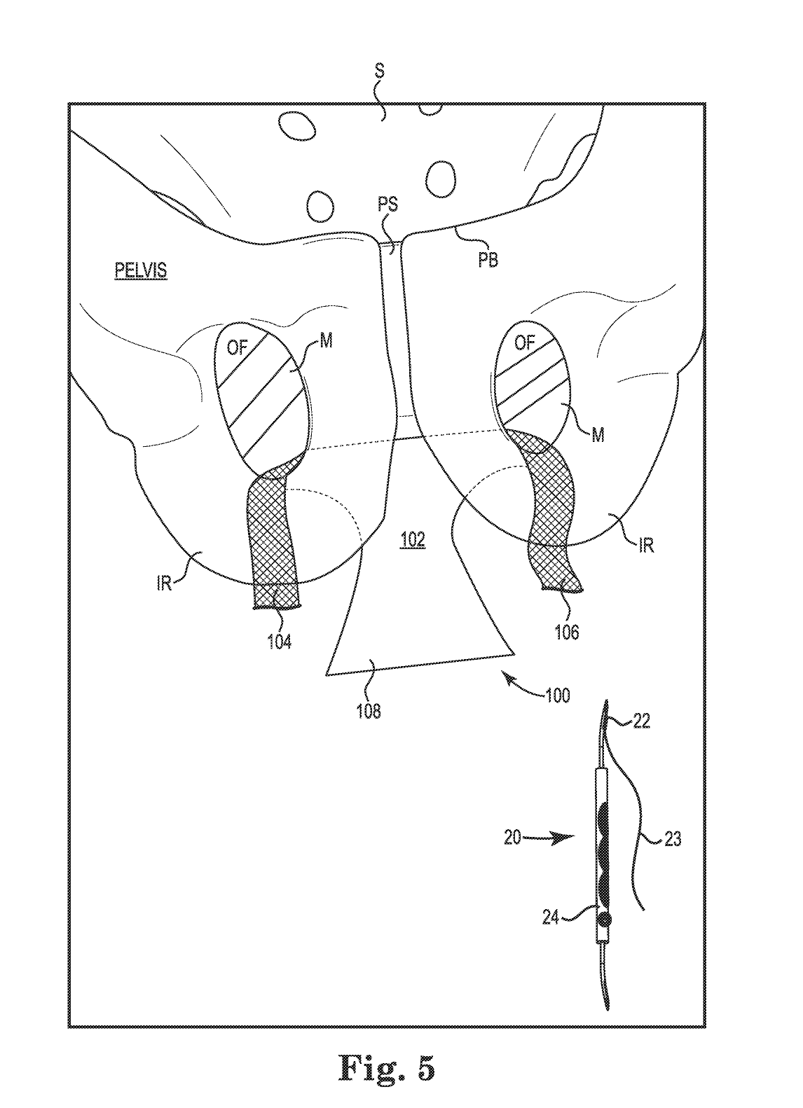

FIG. 5 is a schematic view of one embodiment of a support 100 attachable to a pelvis of a patient. FIG. 5 provides an anterior view of the pelvis with the sacrum S located in a posterior portion of the view, with the pubic symphysis PS centered relative to the pubic bone PB, and an obturator foramen OF on each bilateral side of the pelvis. Each obturator foramen OF provides an opening or a window that is covered by a membrane M. Nerves and arteries traverse the upper reaches of the obturator foramen OF. The membrane M generally includes several layers of muscle and at least one layer of ligament-like tissue that connects the muscles in the membrane M to the pelvis. The ischial pubic ramus IR is located inferior to the pubic bone PB and the obturator foramen OF.

The support 100 is provided to elevate and compress the male urethra and includes a body 102, a first arm 104 extending from the body 102, a second arm 106 extending from the body 102, and a pre-pubic portion 108 that is oriented in a generally orthogonal position relative to the arms 104, 106. The illustrated embodiment is a two-arm device.

Suitable materials for fabricating the support 100 include porous materials that allow tissue ingrowth throughout the support structure to anchor the support 100 in the body after implantation and healing. Suitable such porous materials include autograft material (the patient's own tissue), allograft material (tissue from a cadaver), xenograft material (tissue from another species), or synthetic materials such as woven fabrics, meshes, nonwoven fabrics, meshes, fibrillated fibers, or spun and fibrillated fibers that are provided with voids (pores) configured to allow tissue ingrowth into the support 100. The pores are generally larger, on average, than 75 μm.

The support 100 is attached to the pelvis with each arm 104, 106 inserted into one of the respective obturator foramen OF, and with the pre-pubic portion 108 attached to the periosteum tissue that lines the exterior of the pubic bone PB. The following surgical procedure is one example of the suitable implantation of the support 100 into a male patient.

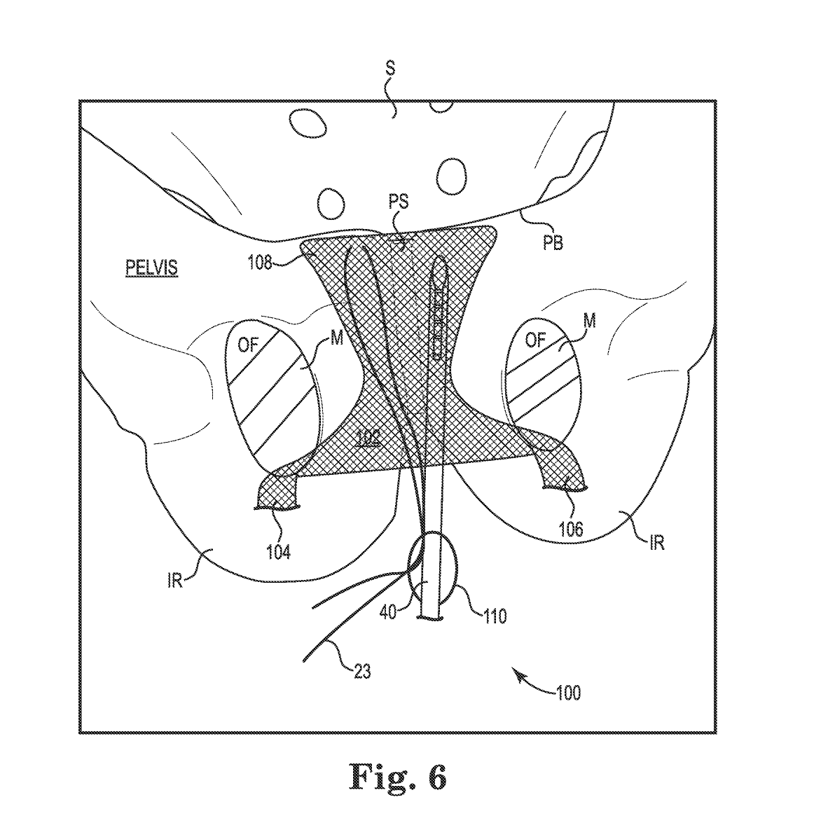

The patient is positioned on a surgical operating table in a lithotomy, or modified lithotomy position, and is anesthetized. A vertical midline perineal incision 110 (see FIG. 6) is formed between the scrotum and the anus. Tissue is dissected to expose the bulbous muscle around the urethra. A suitable tool is used to direct the arm 104 into and through the first obturator foramen OF, and this procedure is repeated on the contralateral side to place the arm 106 into and through the second obturator foramen OF.

One suitable approach of placing the arms 104, 106 through the obturator foramen OF is described as an “outside-in” approach. The outside-in approach includes directing a needle or other device through the skin of the groin area of the patient external of the obturator foramen OF along a curved path through the membrane M and around the ischial pubic ramus R such that the tool exits the midline perineal incision 110. One of the arms 104, 106 is attached to the tool, and the tool is withdrawn along its curved pathway back around the ischial pubic ramus IR, through the membrane M, out of the obturator foramen OF, and out of the skin at the groin area. In this manner, each arm 104, 106 is directed through and placed in one of the obturator foramen OF. The arms 104, 106 are trimmed to a subcutaneous level. A holding stitch is placed to hold the arm 104, 106 relative to the groin tissue, as determined by the surgeon.

A different approach is the “inside-out” approach in which the needle or tool is coupled to the support and directed from the perineal incision (inside) outward to the skin at the groin area (outside). Placement of the arms 104, 106 with the inside-out approach is also acceptable.

One acceptable single incision approach includes the formation of a single (exactly one) incision in the urogenital triangle. Tissue is dissected distal the incision to access the urethra and the pelvis. The arms 104, 106 of the support 100 are directed into the single incision and anchored to the membrane M of the obturator foramen OF, for example with the anchor 22 (FIG. 1). The pre-pubic portion 108 is inserted into the single incision and fixed to the periosteum tissue over the pubic bone PB by the anchor 22 as delivered by the introducer 24. In this manner, a treatment for urinary incontinence is provided to the patient by forming exactly and only one incision and implanting the support 100 through that single incision.

FIG. 6 is a schematic view of the surgical system 20 employed to fixate the pre-pubic portion 108 of the support 100 to the periosteum tissue of the pubic bone PB. The cannula 40 of the introducer 24 is inserted into the perineal incision 110 and directed to the pubic bone PB anterior to the pelvis.

In one suitable approach, the anchor 22 is driven through the material of the support 100 and into the periosteum tissue that covers the pubic bone PB. The cannula 40 pierces the periosteum tissue and slides along the bone of the pelvis without entering or penetrating the bone. The anchor 22 is engaged under the periosteum tissue and the suture 23 extends through the support 100 out through the perineal incision 110. The surgeon, depending upon surgeon preference, will place at least one anchor 22 through the pre-pubic portion 108 an each side of the pubic symphysis PS. The suture 23 extends from each anchor out through the perineal incision 110 and is available for subsequent tying or other termination.

In a different suitable approach, the anchor 22 is loaded into the introducer 24 and the cannula 40 is introduced in the perineal incision 110 up to the pubic bone PB anterior to the pelvis. The introducer 24 is employed to drive the anchor 22 under the periosteum tissue of the pubic bone PB and the cannula 40 is withdrawn through the perineal incision 110. The suture 23 trails behind the anchor 22 and exits the body at the incision 110. An end of the suture 23 is inserted through the pre-pubic portion 108 of the support 100, and the pre-pubic portion 108 is guided along the suture 23, through the incision 110, and up to the pubic bone PB. Thereafter, the suture 23 is tied or terminated to hold the pre-pubic portion 108 against the pubic bone.

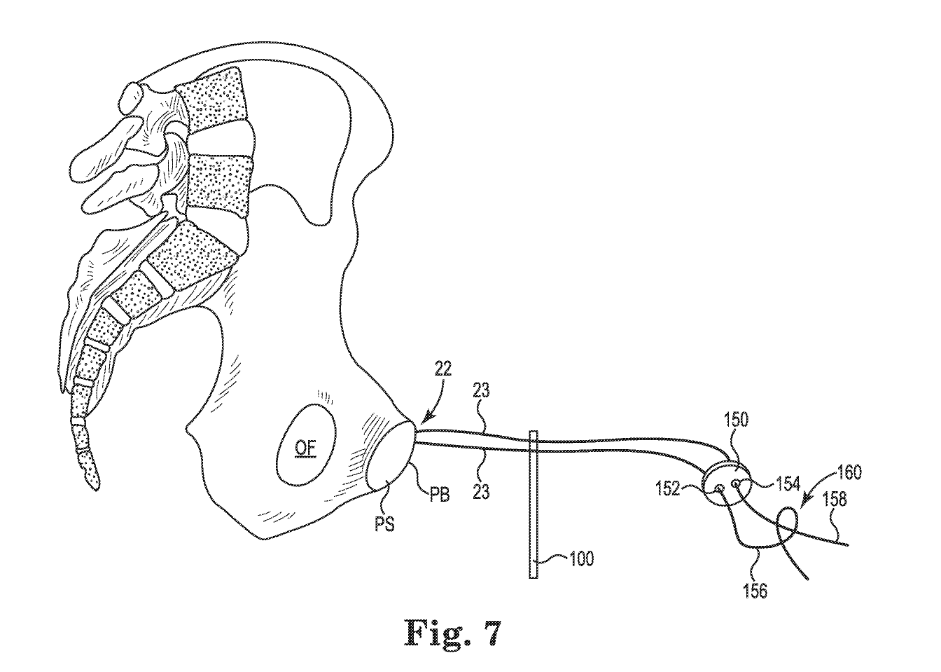

FIG. 7 is a schematic view of the anchor 22 secured to the periosteum tissue and the support 100 secured to the suture 23. In one embodiment, the system 20 described above includes a stopper 150 that is attached to the suture 23, where the stopper 150 is configured to slide along the suture 23 and direct the support 100 into the patient's body and against the tissue. In one embodiment, the stopper 150 has a first orifice 152 and a second orifice 154. One or more of the anchors 22 is engaged with the periosteum tissue of the pubic bone PB, and a first end 156 of the suture 23 extends from the anchor 22 through the first orifice 152, and a second end 158 of the suture 23 extends to the second orifice 154. The stopper 150 slides along the suture 23 and is operable to push or otherwise deliver the support 100 against the pubic bone PB. In one embodiment, a slip knot 160 or other termination device is provided to tie the suture 23 against the stopper 150 after the stopper 150 and the support 100 has been delivered to the pubic bone PB. The stopper 150 is located between the anchor 22 and the slip knot 160.

Suitable materials for fabricating the stopper 150 include plastics or metal. One suitable material for fabricating the stopper 150 includes polypropylene. Another suitable material for fabricating the stopper 150 includes stainless steel. In one embodiment, the stopper 150 is fabricated to be bioabsorbable.

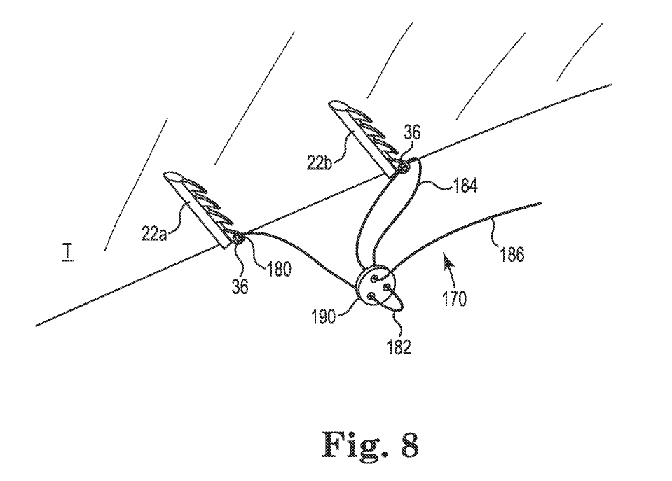

FIG. 8 is a schematic view of two anchors 22 secured to tissue T and coupled with a suture 170. The anchors include a first anchor 22a and a second anchor 22b. The anchors 22 are engaged with the tissue T, for example through the use of the introducer 24 (FIG. 1). A suture 170 is provided having a first end 180 terminated to the eyelet 36 of the anchor 22a, a mid-portion 182 of the suture located between the first anchor 22a and the second anchor 22b, and a portion 184 of the suture in sliding engagement with the eyelet 36 of the second anchor 22b. A free end 186 of the suture 170 is provided, and pulling on the free end 186 of the suture 170 cinches the mid-portion 182 of the suture between the first anchor 22a and the second anchor 22b. In one embodiment, a slide knot 190 or sliding engagement feature 190 is coupled to the suture 170 and is so configured to secure or lock the mid-portion 182 of the suture in a desired position relative to the anchors 22. The slide knot 190 operates to cinch the suture 170 tightly against the support 100 (FIG. 6) against the tissue T.

Some male incontinence treatment devices have several arms, including some form of arms that traverse the obturator foramen and other arms that are implanted anterior to the pubic bone (called pre-pubic arms). The pre-pubic arms are tunneled anterior to the pelvis and exit the skin of the abdomen.

In contrast, embodiments of the system described above provide a support with two arms that are A) secured to the periosteum alongside the obturator foramen or B) secured to the membrane M covering the obturator foramen or C) secured through the obturator foramen and a system 20 to attach a portion of the support directly and efficiently to the periosteum tissue over the pubic bone. The system obviates the use of additional pre-pubic arms that are tunneled under and affixed to the skin. The system is easier to implant and reduces the amount of time that the patient is in the operating room.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of medical devices as discussed herein. Therefore, it is intended that this invention is limited only by its claims and their equivalents.

What is claimed is:

1. A surgical system comprising:
an anchor; and
an introducer provided to deliver the anchor into tissue;
wherein the anchor includes a body having a pointed leading end that is configured to pierce the tissue, a spine projecting radially away from the body and having a width and a height configured to allow the spine to engage with the tissue, an eyelet attached to a trailing end of the body, and a length of suture attached to the eyelet;
wherein the introducer includes a cannula, the cannula having a pointed distal end, an opening formed in the cannula that is sized to receive the body of the anchor, and a slot formed through a wall of the cannula at a distal end portion of the cannula and sized to receive the width of the spine;
wherein when readied for use, the anchor is secured in the introducer with the body of the anchor inserted into the opening of the cannula with the pointed leading end of the body located proximal of the pointed distal end of the cannula, and with the spine of the anchor inserted into the slot of the cannula such that the anchor does not rotate relative to the cannula.

2. The surgical system of claim 1, wherein the body of the anchor is substantially circular in lateral cross-section.

3. The surgical system of claim 2, wherein the body of the anchor has a diameter and the width of the spine is less than 25% of the diameter.

4. The surgical system of claim 1, comprising a plurality of spines projecting radially away from the body, each spine shaped as a shark fin with a curved leading edge meeting with a curved trailing edge at a point, with the curved leading edge diverging away from the pointed leading end of the body.

5. The surgical system of claim 1, wherein the eyelet is disposed on a central longitudinal axis of the body.

6. The surgical system of claim 1, wherein the eyelet projects radially away from a center axis of the body and is so configured to engage with the tissue, and the eyelet has a width substantially equal to the width of the spine.

7. The surgical system of claim 6, wherein the eyelet has a height that is substantially equal to the height of the spine.

8. The surgical system of claim 7, wherein the cannula has a cannula diameter and the height of the spine is at least 50% greater than the cannula diameter.

9. The surgical system of claim 1, wherein the introducer has a handle having a first end opposite a second end and the cannula includes a first cannula extending from the first end of the handle and a second cannula extending from the second end of the handle.

10. The surgical system of claim 1, further comprising:
a stopper attached to the length of suture;
wherein the length of suture includes first strand inserted into a first orifice of the stopper and a second strand inserted into a second orifice of the stopper.

11. The surgical system of claim 10, further comprising:
a slip knot tied in the first and second strands of the length of suture, with the stopper located between the anchor and the slip knot.

12. The surgical system of claim 10, further comprising:
a porous support material, the anchor insertable through the porous support material to locate the anchor on a first side of the porous support material and to locate the stopper on a second opposite side of the first side of the porous support material.

13. The surgical system of claim 1, comprising a first anchor having a first eyelet and a second anchor having a second eyelet, with a second end of the length of suture fixed to the second eyelet of the second anchor, a mid-portion of the length of suture located between the first and second anchors, and the length of suture movable in sliding engagement with the first eyelet of the first anchor.

14. The surgical system of claim 13, further comprising:
a slip knot formed from tying a first end portion of the length of suture to the mid-portion of the length of suture;
wherein the slip knot operates to cinch the length of suture tight against a support that is secured to the tissue by the first and second anchors.

15. A surgical system adapted to fixate a support material inside a human body, the system comprising:
an anchor including a body having a pointed leading end that is configured to pierce tissue, a spine projecting radially away from the body that is configured to engage with the tissue, and an eyelet projecting radially away from the body that is configured to engage with the tissue;
a suture attached to the eyelet; and
an introducer including a cannula having a sharp distal end portion and a slot formed through a wall of the cannula to communicate with a lumen of the cannula, where the slot includes a longitudinal side edge connected between the sharp distal end portion and a proximal lateral edge;
wherein the body of the anchor is sized to fit in the lumen of the cannula such that both the spine and the eyelet extend out of the slot of the cannula;
wherein the proximal lateral edge of the slot is adapted to apply force to the eyelet of the anchor to direct the anchor through the support material and push the anchor into tissue.

16. The system of claim 15, wherein the introducer is configured to locate the anchor on a first side of the support material and locate the stopper on a second side of the support material opposite from the first side of the support material.

17. The system of claim 15, wherein the slot includes a pair of longitudinal side edges that combine to prevent the anchor from rotating within the cannula.

18. The system of claim 15, wherein the suture includes a first strand in sliding engagement with a first orifice of the stopper and a second strand in sliding engagement with a second orifice of the stopper, with the first and second strands looped through the eyelet distal of the stopper and tied to form a knot proximal of the stopper.
